(19) United States
(12) Patent Application Publication
(10) Pub. No.: US 2023/0284650 A1
(43) Pub. Date: Sep. 14, 2023

(54) Soluble Protein Compositions And Methods Of Their Making

(71) Applicant: International Dehydrated Foods, Inc., Springfield, MO (US)

(72) Inventors: Roger Lynn Dake, Springfield, MO (US); Stephanie Lynch, Springfield, MO (US)

(21) Appl. No.: 18/141,288

(22) Filed: Apr. 28, 2023

Related U.S. Application Data
(62) Division of application No. 14/698,332, filed on Apr. 28, 2015, now abandoned.
(60) Provisional application No. 61/985,252, filed on Apr. 28, 2014.

(51) Int. Cl. A23J 1/02; A23L 2/66; A23L 33/17

(52) U.S. Cl. CPC A23J 1/02; A23L 2/66; A23L 33/17

(57) ABSTRACT

This disclosure provides a high quality soluble protein composition and the processes of making the same. The compositions are shelf-stable, easy to use and have excellent nutritional values as compared to other protein products. The compositions may be prepared from animal sources, such as chickens or turkeys.

### RELATED APPLICATION

This application is a Divisional application of U.S. patent application Ser. No. 14/698,332 filed Apr. 28, 2015, which claims priority to U.S. Patent application 61/985,252 filed Apr. 28, 2014, the entire content of all of the above-mentioned applications is hereby incorporated by reference into this application.

### BACKGROUND

### 1. Field of the Invention

This disclosure relates to a process for preparing a complete and soluble protein composition from animal meat or other animal parts. More particularly, the disclosure pertains to soluble protein compositions prepared from poultry and methods of making the same.

### 2. Description of Related Art

Protein is an essential nutrient for humans and animals. Traditional sources of proteins include, for example, animal meat (e.g., poultry, beef, pork, fish, etc.) that are complete in essential and indispensable amino acids; and various plants (e.g., soybean or pea) that are also rich in protein. Convenience foods such as bars, shakes, and smoothies are becoming more and more popular and have become important sources of protein intake. Other protein-rich products such as protein shakes, are used by athletes to maintain or grow muscle mass.

Protein powders are used extensively in making these convenience foods or specialty products. To date, most protein powders are obtained from plant sources, such as soy or pea. Other than protein powders prepared from milk, whey, or eggs, high-protein powders from other animal sources have not been reported or used in such protein-rich products.

### SUMMARY

The disclosed instrumentalities advance the art by providing methods for preparing a high quality soluble protein composition from animal sources. Examples of animal sources may include but are not limited to meat or other body parts from birds, cattle, pigs, among others. Examples of birds may include but are not limited to chickens or turkeys.

The disclosed process improves upon existing processing steps in a unique way. In one embodiment, one or more enzymes may be used in the process of making the soluble protein compositions. In another embodiment, a pressure cooking step may be employed in the disclosed process. It has been unexpectedly discovered that the disclosed process produces higher quality proteins by improving the amino acid composition and ratio extracted from normal raw materials. In one embodiment, the soluble protein composition may contain more than 70%, 80%, 85%, or 90% (w/w) of protein but less than 5%, 3%, 2%, 1%, or 0.5% (w/w) of fat. In another embodiment, more than 70%, 80%, 90%, 95% or 99% (w/w) of the proteins in the composition is soluble in water. In another embodiment, more than 70%, 80%, 90%, 95% or 99% (w/w) of the proteins in the composition is soluble in water at room temperature and under normal pressure. In another embodiment, more than 70%, 80%, 90%, 95% or 99% (w/w) of the proteins in the composition is soluble in water after being heated to a temperature of at least 180 F, or 200 F.

In one embodiment, the instant disclosure provides a soluble protein composition from an animal source. In another embodiment, the product of this disclosure provides a high quality, complete animal protein powder from poultry that is completely soluble. Soluble protein is valuable for applications in beverages and other foods where particulates are not desired. Soluble protein is also advantageous in preparation of special foods that must be administered through tube feeding.

In another embodiment, the percentage of tryptophan by weight of total amino acids in the disclosed soluble protein composition is greater than 0.6%, 0.7%, or 0.8%. In another embodiment, the percentage of hydroxyproline by weight of total amino acids in the disclosed soluble protein composition is less than 2% or less than 3%.

The composition may be prepared from a starting material derived from an animal source. For instance, the starting material may be derived from chicken, turkey, beef, pork or other animal or poultry sources.

In one embodiment, examples of starting material may include but are not limited to meat, boneless meat or poultry trims from chickens or turkeys. In another embodiment, the starting material may be unground poultry parts or carcasses, or ready to cook dressed whole chickens.

In another embodiment, the starting material may be in a substantially liquid form which contains significant amount of poultry sarcoplasm. The term “substantially liquid form” means that the starting material is mostly liquid but may contain minor amount of insoluble material. For instance, the starting material may be in the form of a raw liquid collected from a poultry processing plant or from a poultry storage container or package. In another embodiment, the starting material typically exudes from cut and exposed muscle or bone tissues. This starting material is also known as muscle serum or myogen. In another embodiment, the starting material may appear reddish because it may contain intercellular, and/or intracellular liquid, sarcoplasm, and/or sarcoplasmic reticulum with its proteins, minerals, and metabolites.

In another embodiment, the starting material may be obtained by extracting raw mechanically separated poultry (MSP), mechanically separated chicken (MSC), or finely ground poultry pieces (such as poultry trims or ground poultry parts) with water at room temperature or lower. By way of example, the extraction may be conducted by adding water into raw MSC. The mixture can then be stirred to facilitate mixing and extracting. The ratio between the MSC and water in the extraction mixture may range from about 4:1 to about 1:20 by weight, from about 1:1 to about 1:4 by weight, or about 1:2 by weight. In another embodiment, the MSC and water mixture may be subject to centrifugation at the end of the extraction. The liquid phase resulting from the centrifugation may be collected and used as the starting material for preparing the pumpable broth composition of the present disclosure. In one aspect of this disclosure, the centrifugation may be performed at a speed of at least 1,000 rpm, 2,000 rpm, or at least 3,500 rpm.

The starting material may be prepared on-site and may be used for making the present composition right after it is made fresh on-site. Alternatively, the starting material may be from packaged products or may be collected off-site.

In one aspect, the starting material may be processed (for example, through mechanical grinding) to generate a processed material in the form of fine particles or powders. In another aspect, the starting material may be incubated with one or more enzymes at a temperature between about 100° F. and 160° F., or between 120° F. and 140° F. for a time period between 0.1 to 12 hours, or between 1 and 6 hours, or between 1 and 3 hours, or about 2 hours. In one aspect, the one or more enzymes in this incubation step (a) may be a protease. In another aspect, the one or more enzymes in this incubation step (a) may have a working concentration in the range of 0.1%-0.5%, or about 0.2% by weight.

In another embodiment, the method may include a heating step (b) wherein the mixture from step (a) may be heated to at least 180° F. for at least five minutes to fully cook the mixture and denature the enzymes and form a cooked slurry. In another embodiment, the cooked slurry of step (b) may be further cooked (or incubated) in a step (c) at a temperature between about 200° F. and 300° F. under a pressure between 0 and 60 psig, or between 10 and 15 psig, for a time period between 0.1 and 12 hours, between 1 and 6 hours, or between 1 and 3 hours, to form a fully cooked slurry. In another embodiment, the fully cooked slurry of step (c) may be separated (for example, by centrifugation) in step (d) into at least a fat layer, a broth layer and a solid layer, and the broth layer may be collected in step (e) as the soluble protein composition. In one aspect, the broth layer may contain certain amount of the denatured enzyme. In another aspect, the majority of the denatured enzyme is in the solid layer.

In another embodiment, the method may further contain an acidification step (f) to reduce the pH of the soluble protein composition obtained from step (e). In another embodiment, the acidification step (f) may include adding an acidic agent to the broth layer obtained from step (e) or partially hydrolyzing the broth layer, wherein the acidic agent is selected from the group consisting of carbonated water, carbon dioxide gas, and combination thereof In one aspect, acid hydrolysis may help removing fat (lipid) from the composition. In another aspect, acid hydrolysis may facilitate selective fractionation of proteins and improve amino acid profiles, especially to increase the content of essential amino acids.

In another embodiment, the method may further contain a microfiltration step (g), wherein the microfiltration step selectively enriches one or more amino acids or one or more proteins in the composition obtained from step (e). By way of example, the microfiltration step may be performed by passing the product solution through a membrane filter system designed to either remove or retain selected proteins and/or amino acids.

In another embodiment, the disclosed process may be used to obtain a first protein composition and a second protein composition from the starting material, wherein the first protein composition is liquid and the second protein composition is solid. The fully cooked slurry of step (c) described above may be separated (for example, by centrifugation) into a liquid fraction and a solid fraction. The liquid fraction may be collected as a first protein composition and the solid fraction may be collected as a second protein composition. In another embodiment, the liquid fraction may contain at least a fat layer and a broth layer and only the broth layer is collected as the first protein composition.

The soluble protein composition thus obtained may be used in numerous products, such as, by way of example, protein drink, smoothies, or other nutritional beverages.

In another embodiment, the disclosed high protein, low fat compositions may contain high quality soluble proteins comparable to those of egg proteins. In one aspect, the disclosed composition may have a Protein Efficiency Ratio (PER) score of greater than 60, 70, 80, 85, 90, 95 or 100 as compared to egg protein on PER tests performed on rats. In another aspect, the composition may contain amino acids having an amino acid profile that scores at least 80, 95, 98, or 99 Protein Digestibility Corrected Amino Acid Score (PDCAAS). In another aspect, the composition may contain amino acids having an amino acid profile that scores a perfect 100 Protein Digestibility Corrected Amino Acid Score (PDCAAS). In another aspect, the composition may contain essential amino acids having an amino acid profile that scores higher in a Digestible Indispensable Amino Acid Score (DIAAS) than regular broths prepared from chicken without using enzyme and/or without a pressure cooking step.

In another embodiment, the composition of the present disclosure may be used as a dietary supplement or may be used as a component of a dietary supplement. The supplement may serve various functionalities when administered to a mammal. These functionalities may include but are not limited to promoting growth of certain gut bacterium in the mammal, maintaining a specific gut microbiome, enhancing immunoresponse, modulating inflammatory response of the mammal, or combination thereof. In one particular aspect, the disclosed composition may be used as a prebiotic that promotes gut microbiome or helps balance the different bacterial species in the gut of a mammal. The term microbiome refers to the ecological community of commensal, symbiotic, and pathogenic microorganisms that share a body space within a mammal.

### DETAILED DESCRIPTION

This disclosure relates to a process for making a complete, high quality soluble protein composition from an animal source, such as poultry (e.g., chicken or turkey). In one aspect, the proteins in the disclosed compositions are soluble in water. The disclosed composition may be used by food processors and consumers in a convenient form, for example, as a shelf-stable powder. In another aspect, the amino acid profile offered by this composition is well balanced and obtains excellent scores for PDCAAS.

In one embodiment, the disclosure provides methods of making a high quality protein composition from chicken. Chicken is widely consumed in numerous applications as a healthy, nutritious food. Chicken broth is also widely used as the foundation for many classic foods including soups, stews, chowders, gravies, and sauces. More recently, chicken broth has been used widely in meal kits such as stir fry. However, chicken broth has historically scored lower than insoluble meat proteins in feeding studies, with lower digestibility and PER scores. The PER or Protein Efficiency Ratio score may be derived from growth weight of an animal fed a measured weight of food containing that protein as the only protein in the ration.

As compared to other broth products, the disclosed compositions provide a better balance of amino acids and are a better source of proteins than regular broth prepared according to conventional methods.

Based on Protein Efficiency Ratio testing results, the soluble protein composition disclosed herein is comparable to the highest quality proteins such as egg, and in many cases even exceeds the scores of whey and soy proteins.

Table 1 shows the abundance of amino acids in one exemplary soluble protein composition. Weight percentages of amino acids in the composition are shown in Solids.

TABLE 1
Amino acid profile of the disclosed composition on Solids basis

| | Disclosed |
|---|---|
| | composition |
| | % of |
| | AA |
| Aspartic Acid (w/w) | 8.45 |
| Threonine (w/w) | 4.14 |
| Serine (w/w) | 3.90 |
| Glutamic Acid (w/w) | 15.97 |
| Glycine (w/w) | 6.00 |
| Alanine (w/w) | 5.93 |
| Valine (w/w) | 4.10 |
| Methionine (w/w) | 2.38 |
| Isoleucine (w/w) | 4.03 |
| Leucine (w/w) | 7.24 |
| Tyrosine (w/w) | 2.79 |
| Phenylalanine (w/w) | 6.17 |
| Histidine (w/w) | 2.59 |
| Lysine (w/w) | 8.03 |
| Arginine (w/w) | 6.38 |
| Proline (w/w) | 4.69 |
| Hydroxyproline (w/w) | 1.62 |
| Cysteine (w/w) | 0.41 |
| Tryptophan (w/w) | 0.83 |
| Total | 95.66 |

In another aspect, the amino acid profile of the disclosed soluble protein composition is well balanced. Table 2 shows amino acid profiles of one exemplary soluble protein composition prepared according to the present disclosure. In a study to evaluate the quality of proteins based on amino acid requirements by humans and on protein digestibility, the disclosed protein composition is shown to have a high score (for example, 90) for Protein Digestibility Corrected Amino Acid Score (“PDCAAS”).

TABLE 2
Amino acid profile of the disclosed composition

| | Disclosed | Commercial |
|---|---|---|
| | composition | broth |
| | % of | % of |
| | AA total | AA total |
| Aspartic Acid (w/w) | 8.83 | 6.88 |
| Threonine (w/w) | 4.33 | 2.39 |
| Serine (w/w) | 4.07 | 3.06 |
| Glutamic Acid (w/w) | 16.69 | 14.96 |
| Glycine (w/w) | 6.27 | 17.28 |
| Alanine (w/w) | 6.20 | 8.35 |
| Valine (w/w) | 4.29 | 2.84 |
| Methionine (w/w) | 2.49 | 1.47 |
| Isoleucine (w/w) | 4.22 | 2.10 |
| Leucine (w/w) | 7.57 | 4.56 |
| Tyrosine (w/w) | 2.92 | 1.27 |
| Phenylalanine (w/w) | 6.45 | 2.03 |
| Histidine (w/w) | 2.70 | 2.17 |
| Lysine (w/w) | 8.40 | 4.73 |
| Arginine (w/w) | 6.67 | 7.05 |
| Proline (w/w) | 4.90 | 10.49 |
| Hydroxyproline (w/w) | 1.69 | 7.76 |
| Cysteine (w/w) | 0.43 | 0.43 |
| Tryptophan (w/w) | 0.87 | 0.20 |
| | 100.00 | 100 |

Table 3 shows the typical amino acid composition in total chicken meat protein (USDA SR-21 released Dec. 7, 2011 by U.S. Department of Agriculture).

TABLE 3
Typical amino acid composition of meat protein from chickens

| | Weight |
|---|---|
| | (mg) |
| Aspartic Acid | 3870 |
| Threonine | 1834 |
| Serine (w/w) | 1494 |
| Glutamic Acid | 6504 |
| Glycine | 2133 |
| Alanine | 2369 |
| Valine | 2155 |
| Methionine | 1203 |
| Isoleucine | 2293 |
| Leucine | 3259 |
| Tyrosine | 1466 |
| Phenylalanine | 1724 |
| Histidine | 1348 |
| Lysine | 3689 |
| Arginine | 2619 |
| Proline | 1785 |
| Hydroxyproline | ND |
| Cysteine | 556 |
| Tryptophan | 507 |
| Total | 43400 |

Table 4 shows the content of metals and minerals in one exemplary soluble protein composition prepared according to the present disclosure.

TABLE 4
Metal and Mineral content of the
disclosed composition from chickens

| | Amount | |
|---|---|---|
| Aluminum | <10 | ppm |
| Calcium | 0.03% | |
| Chromium | <1.2 | ppm |
| Copper | 1 | ppm |
| Iron | 0.0011% | |
| Magnesium | 0.0824% | |
| Manganese | <0.5 | ppm |
| Nickel | <0.7 | ppm |
| Phosphorus | 0.71% | |
| Potassium | 1.58% | |
| Selenium | 0.72 | ppm |
| Sodium | 0.64% | |
| Zinc | 14 | ppm |

In one embodiment, the disclosed soluble composition contains significantly higher concentration of tryptophan (greater than 0.6% of total amino acids) as compared to commercially available broth (about 0.1-0.3%). See Table 2. The use of enzyme in the present process may help render more tryptophan-containing proteins (or peptides) soluble. In another aspect, the amount of hydroxyproline is relatively low (e.g., 1.69% in one embodiment) in one broth composition as compared to commercially available broth (e.g., 7.76%).

In another embodiment, because the disclosed soluble protein compositions provide complete amino acid profile in a single high quality soluble protein source, there is no need to mix multiple protein sources or to supplement any particular amino acids. Indeed, as shown by Protein Efficiency Ratio (PER) testing, the soluble protein compositions of the present disclosure are comparable to the highest quality proteins such as those from eggs, while in many cases, exceeding the scores of whey, pea and soy proteins. Protein efficiency ratio (PER) is measured based on the weight gain of a test subject divided by its intake of a particular food protein during the test period.

Due to conditions such as kidney disease, some people must monitor their dietary protein intake closely. For these people, proteins of the highest quality are optimum for sourcing and consumption. In one embodiment, the soluble proteins of the disclosed composition may be easier for kidneys to process than regular digested proteins.

In one aspect, the disclosed process may be used to turn lower value raw poultry materials into a high value protein powder or broth without using additives. By way of example, several representative steps of one of the embodiments of the disclosed processes are described below:

1. In one embodiment, raw chicken muscle such as boneless meat or trims may be used as the starting material. Alternatively, raw chicken frames and carcasses may be converted to mechanically separated chicken using equipment and techniques available in the art.

2. This material may be finely ground to allow optimum fractionation of protein from fat and other non-protein materials. Pieces of the starting raw material may be ground to less than 5 mm, 4 mm, 3 mm, 2 mm, or less than 1 mm size.

3. The finely ground chicken starting material may be mixed with water as needed for easy handling.

4. Protease enzyme(s) may be added to the mixture at an optimum percentage that depends on the enzymes used and the finished product desired. This mixture is heated to an optimum digesting temperature and allowed to react for an optimum time. Process times and temperatures are varied for best results. By way of example, a weight of enzyme equal to 0.2% of the protein content of the raw material may be used, and the temperature may be set at 130° F. for two hours.

5. After the enzyme treatment, the mixture is heated to at least 180° F. for at least five minutes to fully cook the mixture and denature the enzymes. This initial cooking time and temperature may also be varied to achieve desired results.

6. The cooked slurry may be further cooked to optimize the amount of product yielded and optimize the amino acid profile of the finished product. The product may be cooked at very high temperatures and pressures for an extended time if needed. Parameters for this additional cooking may include, for example, at a temperature between 230 and 300° F. for a time period between 1 and 6 hours.

7. After all cooking is completed, the desired soluble protein fraction (broth) is separated from the fat and solids fractions and collected for further processing. It may be collected by various techniques known in the art, which include but are not limited to screening, decanting, settling, and filtering.

8. The collected soluble protein broth may be concentrated for use or sale in liquid versions or dried by methods known in the art to make a powder.

The processing step combination of enzyme digestion and extended cooking of the finely ground product results in a high yield of soluble protein that has shown by amino acid analysis to be of higher quality than regular broth products made simply by plain cooking, regardless of the materials or cooking process used. The combination of enzymatic and thermal processes extract and provide more essential amino acids than those extracted using existing processes.

In one embodiment, the soluble protein compositions may be used as an ingredient in food or beverage products. In another embodiment, the composition obtained may be used in numerous applications as a wholesome, all natural ingredient. The disclosed extract may also be used to prepare protein drinks, smoothies, or other nutritional beverages.

It is to be noted that, as used in this specification and the claims, the singular forms “a,” “an,” and “the” include plural referents unless the context clearly dictates otherwise. Thus, for example, reference to “a device” may include reference to one device, as well as two or more devices, unless the context clearly limits the reference to one device.

The terms “between” and “at least” as used herein are inclusive. For example, a range of “between 5 and 10” means any amount equal to or greater than 5 but equal to or smaller than 10.

Unless otherwise specified, the percentage of certain component in a composition is by weight of total solid. Various commercially available products may have been described or used in this disclosure. It is to be recognized that these products are cited for purpose of illustration only. Certain physical and/or chemical properties and composition of the products may be modified without departing from the spirit of the present disclosure. One of ordinary skill in the art may appreciate that under certain circumstances, it may be more desirable or more convenient to alter the physical and/or chemical characteristics or composition of one or more of these products in order to achieve the same or similar objectives as taught by this disclosure.

### EXAMPLES

The following examples are provided to illustrate the present invention, but are not intended to be limiting. The reagents, materials and instruments are presented as typical components, and various substitutions or modifications may be made in view of the foregoing disclosure by one of skills in the art without departing from the principle and spirit of the present invention.

### Example 1 Preparation of a Soluble Protein Composition From Poultry Using One or More Enzymes

Raw mechanically separated chicken (MSC) was placed into a vessel with an equal amount of water. Two protease enzymes were added at 0.2 percent (by weight) of chicken solids and thoroughly mixed into the raw slurry. The mixture was heated to 130° F. (or about 54.4° C.) and held for 2 hours with intermittent agitation. The mixture was then put into a pressure vessel (or cooker) and cooked at 10-15 psi (240 to 250° F.) for 3 hours. This step helped fully cook the product and completely denatured the enzymes. The cooked slurry was allowed to cool to less than 200° F. for safe handling. Then, the slurry was centrifuged at 3,500 rpm to separate the slurry into fat, broth, and meaty layers. These different layers were separated and collected. The broth layer was concentrated by evaporation to obtain the soluble protein composition.

This soluble protein composition was subject to amino acid analysis. The amino acid profile of this composition was compared to those of other chicken broths, which contained only chicken broth with no seasonings, flavorings, or other protein sources. An PDCAAS calculator was used to calculate the PDCAAS (http://www.unjury.com/protein_tools/pdcaas.html). The soluble protein composition of this Example had a PDCAAS of 100 against egg white as the standard. All other control broths scored less than 60.

A scaled up experiment using the same starting materials and same process was also conducted. The resultant composition after drying contained about 5% moisture, 86.6% protein, 1% fat, and 5.5 ash.

These results show that the combination of enzyme treatment plus pressure cooking extracted a composition having high quality protein and low fat. The composition also has superior ratio of amino acids.

The nutritional scores of the disclosed composition (SPC for soluble protein composition) were also compared to a number of other proteins. Various protein compositions were fed to rats and the efficiency of these different protein compositions was measured according to standard protocols. The results of protein efficiency ratio (PER), relative PER (RPER), net protein ratio (NPR) and relative NPR (RNPR) values are shown in Table 6.

TABLE 6
Comparison of protein quality scores

| | | RPER | | | | | | Limiting |
|---|---|---|---|---|---|---|---|---|
| | PER | casein | Digestibility | NPR | RNPR | PDCAAS | DIASS | AA |
| Casein | 1.93 | 100 | 98.21 | 1.13 | 100 | | | |
| SPC | 1.57 | 90.53 | 96.97 | 1.13 | 92.77 | 100 | 91.10 | Leucine |
| Whey | 1.19 | 61.78 | 97.71 | 0.45 | 38.01 | 100 | 61.40 | Histidine |
| Soy | 1.85 | 95.97 | 97.63 | 1.28 | 104.10 | 100 | 46.4 | Methionine |
| Pea | 0.98 | 50.81 | 97.83 | 0.23 | 16.33 | 80.42 | 36 | Methionine |

### Example 2 Comparison of Soluble Protein Compositions From Poultry Prepared With or Without Using Enzyme

A control experiment was conducted using the same MSC as starting material under the same protocol but without the use of enzymes. The soluble protein composition obtained under this no-enzyme process had a PDCAAS score between 41 and 57 by using the same PDCAAS calculator. All other control broths scored less than 60.

### REFERENCES

All references listed below and those publications, patents, patent applications cited throughout this disclosure are hereby incorporated expressly into this disclosure as if fully reproduced herein.
Shah et al., U.S. patent application No. 10/912,560.
Shah et al., U.S. patent application No. 10/919,518.
Shah et al., U.S. patent application No. 10/932,295.
Shah et al., U.S. patent application No. 10/972,089.
Shah et al., U.S. patent application No. 11/11/153,435.
USDA SR-21 released Dec. 7, 2011 by U.S. Department of Agriculture.

We claim:

1. A method for making a soluble protein composition from a starting material derived from an animal source, said composition comprising one or more soluble proteins, said method comprising:
(a) incubating said starting material with an enzyme at a temperature between about 120° F. and 140° F. for a time period between 1 and 3 hours,
(b) heating the mixture from step (a) to at least 180° F. for at least five minutes to fully cook the mixture and denature the enzymes and form a cooked slurry,
(c) incubating the cooked slurry of step (b) further at a temperature between about 200° F. and 300° F. under a pressure between 10 and 15 psig for a time period between 1 and 6 hours to form a fully cooked slurry,
(d) separating the fully cooked slurry of step (c) into at least a fat layer, a broth layer and a meaty layer, and
(e) collecting said broth layer to obtain said soluble protein composition.

2. The method of claim 1, wherein said separation step (d) is carried out by centrifugation.

3. The method of claim 1, wherein said enzyme in step (a) comprises a protease.

4. The method of claim 3, wherein said protease has a working concentration in the range of 0.01%-1% by weight.

5. The method of claim 3, wherein said protease has a working concentration of about 0.2% by weight.

6. A method for making a first protein composition and a second protein composition from a starting material derived from an animal source, said first protein composition being soluble, and said second protein composition being insoluble, said method comprising:
(a) incubating said starting material with an enzyme at a temperature between about 100° F. and 160° F. for a time period between 0.1 and 12 hours,
(b) heating the mixture from step (a) to at least 180° F. for at least five minutes to fully cook the mixture and denature the enzymes and form a cooked slurry,
(c) incubating the cooked slurry of step (b) further at a temperature between about 200° F. and 300° F. for a time period between 0.1 and 12 hours to form a fully cooked slurry,
(d) separating the fully cooked slurry of step (c) into at least a solid fraction and a liquid fraction, and
(e) collecting said liquid fraction to obtain said first protein composition, and collecting said solid fraction to obtain said second protein composition.

7. The method of claim 6, wherein said liquid fraction comprises a fat layer and a broth layer, said broth layer being collected as said first protein composition.

8. The method of claim 6, wherein said incubating in step (c) is performed under a pressure between 10 and 15 psig.

9. The method of claim 1, further comprising an acidification step (f) to reduce the pH of said soluble protein composition obtained from step (e).

10. The method of claim 9, wherein said acidification step (f) comprises adding an acidic agent to said broth layer obtained from step (e) or partially hydrolyzing said broth layer, wherein said acidic agent is selected from the group consisting of carbonated water, carbon dioxide gas, and combination thereof.

11. The method of claim 1, further comprising a microfiltration step (g), said microfiltration selectively enriching certain proteins or certain amino acids in said composition obtained from step (e).

12. A method of making a food or beverage product, comprising the step of using the soluble protein composition obtained in claim 1 as an ingredient of the food or beverage product.

13. The method of claim 12, wherein said food or beverage product is selected from the group consisting of protein drink, smoothies, and other nutritional beverages.

14. A method of making a dietary supplement, comprising the step of using the soluble protein composition obtained in claim 1 as an ingredient of the dietary supplement.

15. The method of claim 14, wherein said dietary supplement is a prebiotic supplement.
